(19) United States
(12) Patent Application Publication
(10) Pub. No.: US 2011/0275110 A1
(43) Pub. Date: Nov. 10, 2011

(54) METHOD FOR ISOLATING OR COUNTING MICROORGANISMS ON AN AGAR CULTURE MEDIUM

(75) Inventors: Bruno Colin, Marcy L'Etoile (FR); Aurelien Costa, Grenoble (FR)

(73) Assignee: BIOMERIEUX S.A., Marcy l'Etolie (FR)

(21) Appl. No.: 13/143,879

(22) PCT Filed: Jan. 29, 2010
(86) PCT No.: PCT/FR2010/050146

§ 371 Date: Jul. 8, 2011

(30) Foreign Application Priority Data
Jan. 30, 2009 (FR) 0950591

(51) Int. Cl. C12Q 1/24; C12N 1/00

(52) U.S. Cl. 435/29; 435/243

(57) ABSTRACT

The invention relates to a method for isolating microorganisms on an agar culture medium, including the steps of:
depositing, on said agar culture medium, a predetermined amount of a sample to be analyzed and optionally containing said microorganisms or a suspension of said microorganisms;
applying a seeding means onto agar culture medium in contact with the volume of sample or suspension;
moving the seeding means so as totally or partially spread the volume of sample or suspension over the surface of the agar culture medium, the movement of said seeding means being discontinuous so that during said movement the contact between the seeding means and the surface of the agar culture medium is interrupted and re-established at least once, thereby creating spreading segments and resulting in a depletion of the seeding means in terms of the sample or suspension; and
incubating said agar culture medium under conditions enabling the growth of microorganisms.

The field of the invention is that of the analysis of target microorganisms in a complex sample. More particularly, the present invention relates to a method for isolating, or even counting, microorganisms on an agar culture medium, from a liquid sample to be analyzed or from a suspension of microorganisms.

The isolation of microorganisms on an agar culture medium, from a liquid sample to be analyzed or from a suspension of microorganisms, is a step that is often essential for many methods of microbiological analysis. This step is in particular used to carry out identifications, to verify the microbial purity of a sample or else to perform a bacterial count by counting the resulting isolated colonies.

One of the major problems for this bacterial isolation step is related to the ratio between the amount of microorganisms to be spread and the exploitable surface area. This is because most agar medium supports have a surface area which makes it possible to isolate only an amount of bacteria of between 15 and 300 CFU (Colony Forming Units) which give just as many colonies after growth.

In most cases, the sample to be analyzed contains an indeterminate amount of microorganisms that can range from zero to more than 10⁸ bacteria per milliliter (ml). Thus, in order to be sure of efficient isolation, it is necessary, upstream of the spreading on agar culture medium, to carry out a series of cascading dilutions (commonly by a factor of 10) in order to reduce, via successive stages, the microbial load of the sample. A given volume of each dilution thus prepared is then spread on agar medium. The Petri dishes corresponding to each of the dilutions are then incubated in a thermostatic chamber. After microbial growth, it is possible to select the Petri dish of agar medium of which the microbial load on the dish is sufficiently low to distinguish and optionally subculture the isolated colonies.

In the perspective of carrying out a microbial count, it is essential to use a Petri dish comprising only isolated colonies. The result obtained in this way is considered to be reliable when it is comprised between 15 and 300 isolated colonies.

Although effective, the implementation of conventional isolation methods as previously described has the drawback of being very laborious and of consuming a large number of reagents (Petri dishes, tubes of diluents, loops, etc.) generating a high volume of waste (autoclaving, cost of treatment).

Some manual methods have been automated by virtue of the development of devices. This is the case, for example, in document EP-0 242 114, which describes an apparatus and a method for inoculating a culture medium with a sample. The method consists in producing several spreading segments starting from an inoculum. These segments are in the form of an arc of a circle and are produced by means of four different spreading heads. A sample dilution effect is obtained by partial overlapping of the subsequent segments. The method described in the document is in fact very similar to the reference manual isolation method which consists in producing several spreading segments from a single inoculum, with overlapping of the segments in order to load the inoculating means with bacteria and to obtain a depletion of bacteria during the subsequent spreading segment.

Document FR-A-2 694 570 describes a method and a system for depositing bacterial solutions on a culture medium by means of a stylet in fluidic communication, via a pipe, with a bacterial solution distributor driven by means of a jack. The bacterial solution is deposited in the form of spirals or spots, by rotating the culture medium on an ad hoc platform at the same time as the bacterial solution is poured onto the medium. Such a method cannot be considered to be an isolation method insofar as the bacterial solution is poured throughout the rotation of the medium. Specifically, there is no depletion of bacterial solution and therefore of bacteria.

More recently, new isolation methods have seen the light of day, which make it possible to improve the bacterial depletion by using an optimized applicator (WO-A-2005071055). This is in particular the case for the inoculating method used in the automated device sold by the applicant under the reference PREVI™ Isola. The implementation of this new method of isolation makes it possible to obtain isolated colonies from a wider range of microbial load in the initial sample to be analyzed. Despite the improvement provided by the use of this optimized applicator, the constraint related to the microbial load/agar surface area ratio remains real and the use of this technique can find its limits with very highly contaminated samples. Furthermore, this technique does not, at the current time, make it possible to carry out a precise evaluation of the microbial load of the initial sample owing to the proximity of the colonies that are difficult to count.

It emerges from the prior art considered that there is no method for isolating, or even counting, microorganisms which is simple to carry out from a sample to be analyzed or from a bacterial suspension, on a single agar culture medium and which makes it possible to obtain, on a limited surface area of agar, isolated colonies irrespective of the initial bacterial load of said sample or of said suspension.

A first objective of the present invention is therefore to provide a method for isolating microorganisms which is more effective than the prior art methods.

A second objective of the present invention is to provide a method of counting which is more effective than the prior art methods.

A third objective of the present invention is to provide a method for isolating, or even counting, microorganisms which makes it possible to obtain isolated colonies over a very wide range of microorganism loads.

A fourth objective of the present invention is to provide a method for isolating, or even counting, microorganims which makes it possible to obtain a reliable evaluation of the microorganism load of the initial sample or of the initial suspension.

A fifth objective of the present invention is to provide a method for isolating, or even counting, microorganisms which can be exploited on a reduced surface area of agar culture medium.

These objectives, among others, are achieved by the present invention, which relates firstly to a method for isolating microorganisms on an agar culture medium, comprising the steps consisting in:
depositing, on said agar culture medium, a predetermined volume of a sample to be analyzed optionally containing said microorganisms or of a suspension of said microorganisms,
applying an inoculating means onto said agar culture medium in contact with the volume of sample or of suspension,
moving the inoculating means so as to totally or partially spread the volume of sample or of suspension over the surface of the agar culture medium, the horizontal movement of said inoculating means being discontinuous such that, during this movement, the contact between said inoculating means and the surface of the agar culture medium is interrupted and re-established at least once, creating successive spreading segments and resulting in a depletion of the inoculating means in terms of sample or of suspension, and
incubating said agar culture medium under conditions enabling the growth of microorganisms.

A second object of the present invention relates to a method for counting microorganisms on an agar culture medium, comprising the steps consisting in:
depositing, on said agar culture medium, a predetermined volume of a sample to be analyzed optionally containing said microorganisms or of a suspension of said microorganisms,
applying an inoculating means onto said agar culture medium in contact with the volume of sample or of suspension,
moving the inoculating means so as to totally or partially spread the volume of sample or of suspension over the surface of the agar culture medium, the horizontal movement of said inoculating means being discontinuous such that, during this movement, the contact between said inoculating means and the surface of the agar culture medium is interrupted and re-established at least once, creating successive spreading segments and resulting in a depletion of the inoculating means in terms of sample or of suspension,
incubating said agar culture medium under conditions enabling the growth of microorganisms, and
counting the microorganism colonies present at the surface of the agar culture medium.

According to the invention, the interruption and the re-establishment of the contact between the inoculating means and the agar culture medium during the movement of said means can be likened to a jump of said means. This jump allows the inoculating means to be depleted in terms of sample or suspension. This is because, as long as the inoculating means is in contact with the agar culture medium, it carries the liquid along by capillary drainage. When the contact between the inoculating means and the agar culture medium is interrupted, said inoculating means is moved away from the surface of the culture medium, until there is detachment from the liquid. The liquid vein is then no longer carried along.

During this detachment, the inoculating means carries along with it a fraction of sample or of suspension that has remained attached to said inoculating means.

The movement of the inoculating means, while the latter is out of contact with the culture medium, is continued along a direction substantially parallel to the surface of said culture medium. This movement must be sufficient so that, when the contact between the inoculating means and the agar culture medium is re-established, this new point of contact is sufficiently far from the last point of contact before interruption, in order to avoid any contact between the inoculating means and the preceding spreading. This is because such a contact can result in transfer of sample or of suspension from the first spreading, and of the associated bacteria, onto the inoculating means, thus limiting the depletion phenomenon. Moreover, this would amount to carrying out a conventional isolation, in which, during a spreading, the inoculating means intersects the preceding spreadings, in order to be reloaded.

When the contact is re-established, the inoculating means reproduces its horizontal movement on the agar culture medium, carrying along the fraction of sample or of suspension that has remained attached, by capillary drainage, and allowing a new spreading of this fraction.

Several successive jumps of the inoculating means can be carried out, such that, at each interruption of contact, a detachment of liquid occurs, accentuating the depletion of the inoculating means in terms of sample or suspension.

The factors which influence the amount of sample or suspension retained on the inoculating means during the interruption of contact are essentially:
the wettability of the agar culture medium,
the surface tension of the sample or of the suspension.

These two parameters influence the angle of contact between the liquid fraction of sample or of suspension and the surface of the culture medium and therefore the force necessary to interrupt the liquid vein.

These two parameters are in essence variable depending on the type of agar culture medium used, but also the type of sample or of suspension to be analyzed.

Advantageously, in the methods according to the invention, the inoculating means has a multitude of surfaces of contact with said culture medium. Such an inoculating means may be, for example, an applicator used with the PREVI™ Isola system, as protected in patent application WO-A-2005071055.

Alternatively, the inoculating means has a single surface of contact with said culture medium. Such a means may, for example, be a loop, a platinum wire loop or a swab.

The movement of the inoculating means may advantageously be a rectilinear movement. The term “rectilinear movement” is intended to mean a single or several rectilinear segments, optionally in different directions. Such a movement is conventionally used in the conventional method of isolation by means of a loop or a platinum wire loop.

Alternatively, the movement of the inoculating means is a curvilinear movement. Such a movement is that used in the PREVI™ Isola system. Specifically, the movement of the inoculating means follows the edges of the Petri dish when the latter is a round dish. Moreover, when the inoculating means has several surfaces of contact with the culture medium, this curvilinear movement makes it possible to increase the length of the spreading.

Advantageously, the method according to the invention may be implemented by means of an automated system. A particularly suitable system is the PREVI™ Isola system sold by the applicant.

According to one preferential embodiment, the number of times the contact between said inoculating means and the surface of the agar culture medium is interrupted and re-established is between 2 and 6 times.

According to another preferential embodiment, the volume of sample or of suspension deposited on the agar culture medium is between 10 and 1000

According to one particularly advantageous embodiment, the spreading segments are variable in length. This is because, for the same isolation, it may be advantageous to produce successive spreading segments of different lengths. This is in particular the case for samples suspected of being excessively loaded with microorganisms. Several spreading segments of limited length will make it possible to deplete the inoculating means very rapidly, over a very small surface area of culture medium. The subsequent spreading segments are, on the other hand, of greater length in order to make it possible to obtain isolated colonies.

The aims and advantages of the method according to the invention will emerge more clearly on reading the detailed description which follows, in connection with the drawing in which:

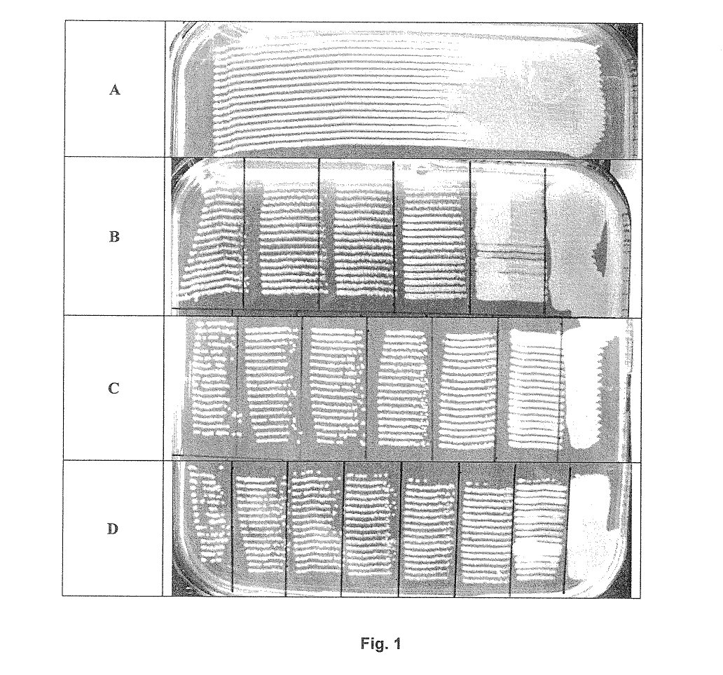

FIG. 1 represents the images of a comparative analysis between an isolation carried out according to the conventional method and isolations carried out according to various procedures of the method of the invention, with a bacterial suspension at approximately 10⁸ CFU/ml.

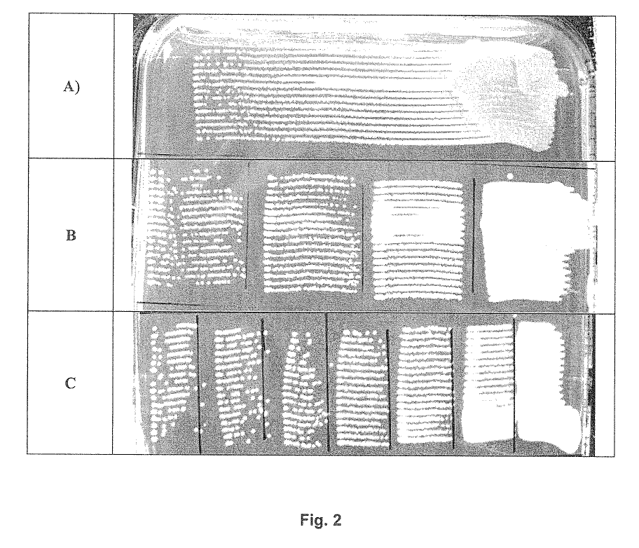

FIG. 2 represents the images of a comparative analysis between an isolation carried out according to the conventional method and isolations carried out according to various procedures of the method of the invention, with a bacterial suspension at approximately 10⁷ CFU/ml.

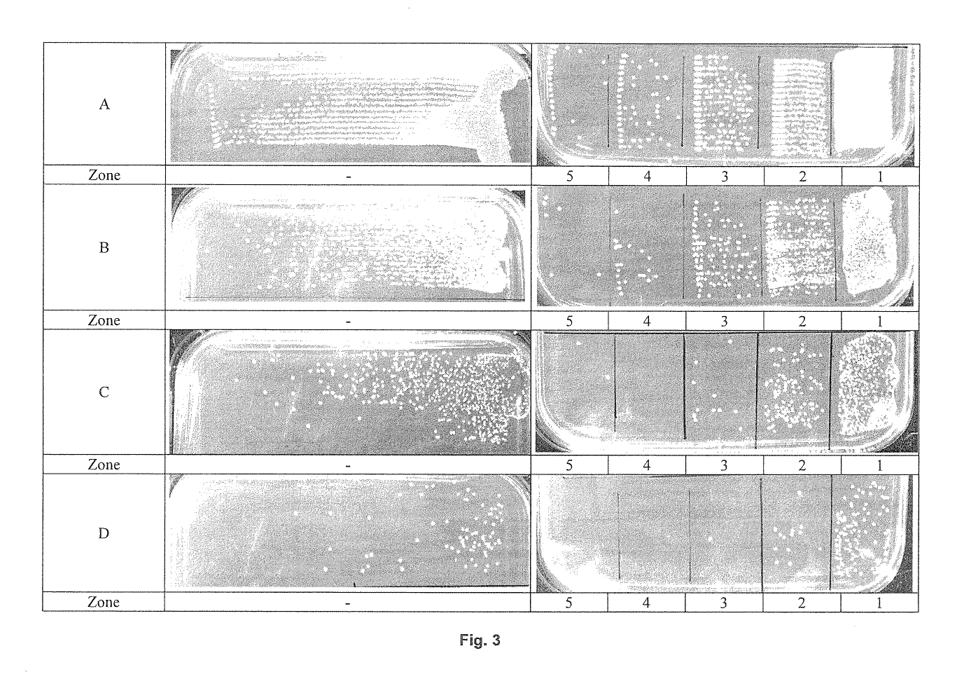

FIG. 3 represents the images of a comparative analysis between an isolation carried out according to the conventional method and an isolation carried out according to the method of the invention, followed by counting of the bacterial colonies, with suspensions having variable bacterial loads.

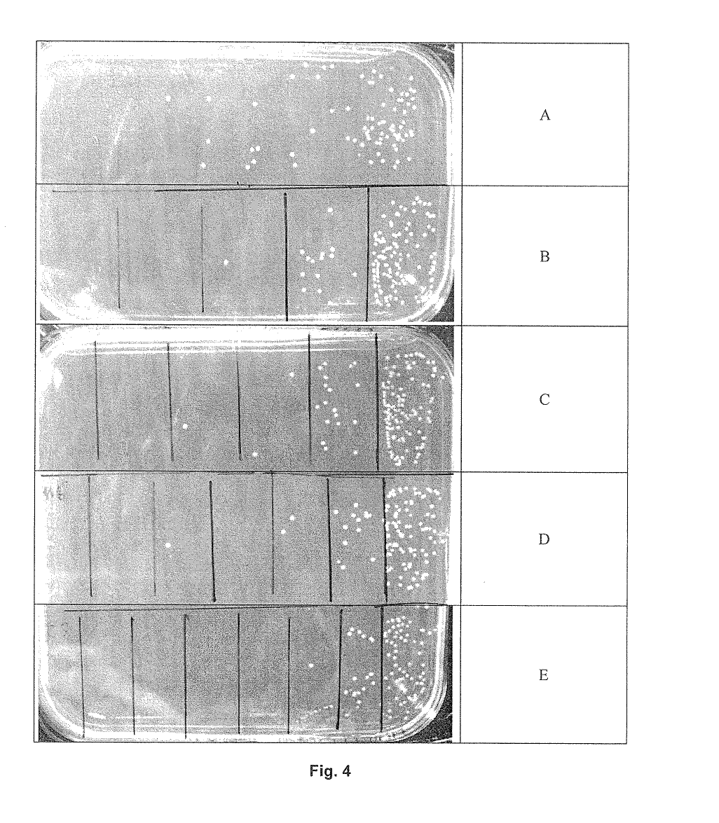

FIG. 4 represents the images of a comparative analysis between counting of bacterial colonies carried out according to the conventional method and counting of bacterial colonies carried out according to the method of the invention, and also the evaluation of the bacterial suspension volume distribution factor after each re-contacting.

### EXAMPLES

### Example 1

### Obtaining of Isolated Colonies from a Highly Contaminated Solution, on a Reduced Surface Area of Agar

### Procedure:

100 μl of a solution highly loaded with Staphylococcus aureus (10⁷ or 10⁸ CFU/ml) are deposited on the edge of a dish of agar culture medium. This volume is then manually spread in a rectilinear manner using a spreading means consisting of the applicator used with the PREVI™ Isola system, as protected in patent application WO-A-2005071055.

A control spreading is carried out without performing any jump with the applicator. Using a deposit identical to the control condition, various spreadings are carried out in parallel with the addition of an increasing number of jumps during the spreading.
FIG. 1A: no jump (suspension at approximately 10⁸ CFU/ml)
FIG. 1B: 5 jumps (suspension at approximately 10⁸ CFU/ml)
FIG. 1C: 6 jumps (suspension at approximately 10⁸ CFU/ml)
FIG. 1D: 7 jumps (suspension at approximately 10⁸ CFU/ml)
FIG. 2A: no jump (suspension at approximately 10⁷ CFU/ml)
FIG. 2B: 3 jumps (suspension at approximately 10⁷ CFU/ml)
FIG. 2C: 6 jumps (suspension at approximately 10⁷ CFU/ml)

After incubation, a search for isolated colonies is carried out and the length of spreading required to obtain these colonies is measured.

### Results:

With regard to the suspension at 10⁸ CFU/ml, the width (10 cm) of the Petri dish is not sufficient to allow isolated colonies to be obtained with spreading by the conventional method, namely by moving the spreading means without performing a jump (FIG. 1A).

When 5 jumps of the spreading means are performed in a regular manner on the culture medium, the first isolated colonies appear after 7.5 cm of spreading length.

When 6 jumps of the spreading means are performed in a regular manner on the culture medium, the first isolated colonies appear after 4.5 cm of spreading length.

When 7 jumps of the spreading means are performed in a regular manner on the culture medium, the first isolated colonies appear after 1.5 cm of spreading length.

With regard to the suspension at 10⁷ CFU/ml, with spreading by the conventional method, the first isolated colonies appear after 7.2 cm of spreading length (FIG. 2A).

When 3 jumps of the spreading means are performed in a regular manner on the culture medium, the first isolated colonies appear after 3 cm of spreading length.

When 6 jumps of the spreading means are performed in a regular manner on the culture medium, the first isolated colonies appear after 4 cm of spreading length.

The results obtained show that, when jumps are performed with the inoculating means during the spreading of the sample on the culture medium, the depletion in terms of bacteria is more effective, thus making it possible to obtain isolated colonies on a reduced surface area of agar culture medium. Moreover, it is noted that, the higher the number of jumps, the shorter the spreading length of the sample required for obtaining isolated colonies.

### Example 2

### Advantages of Jump Spreading for Producing a Model for Reliable Counting of the Microbial Load of a Sample on an Agar Medium

### Procedure:

100 μl of solutions loaded with Staphylococcus aureus at various concentrations obtained by successive 10-fold dilutions are deposited on the edge of an agar culture medium. This volume is then manually spread in a rectilinear manner using the applicator used on the PREVI™ Isola system.

A control spreading is carried out without performing a jump with the applicator. Using a deposit identical to the control condition, spreading comprising 4 successive jumps is performed in parallel, these 4 jumps defining 5 distinct zones, denoted zone 1 to zone 5.

The results obtained are represented in FIG. 3, in which the left-hand column corresponds to the control spreadings and the right-hand column to the spreadings according to the method according to the invention, with successive jumps.

Moreover, row A corresponds to the results obtained with a bacterial load of between 80 000 and 130 000 CFU.

Row B corresponds to the results obtained with a bacterial load of between 8000 and 13 000 CFU.

Row C corresponds to the results obtained with a bacterial load of between 800 and 1300 CFU.

Row D corresponds to the results obtained with a bacterial load of between 80 and 130 CFU.

After incubation of the culture media, counting of the isolated colonies is carried out on each zone delimited by a comb jump performed during the spreading.

### Results:

It appears, firstly, that certain zones cannot be counted owing to the proximity of the nonisolated colonies.

The control results (without jump) show that, with conventional spreading, counting can only be performed with the lowest bacterial load (of about 100 CFU). Thus, as can be seen, in FIG. 3, row D, left-hand column, approximately 80 colonies are isolated.

The optimization of the spreading, with jumps (4 in this example), makes it possible, with the same initial load, to clearly delimited several zones, some of which display only isolated colonies which can therefore be counted.

Thus, in row A, right-hand column, zones 4 and 5 show a count of 67 and 34 isolated colonies, respectively.

In row B, zones 3, 4 and 5 show a count of 116, 24 and 6 isolated colonies, respectively.

In row C, zones 2, 3 and 5 show a count of 113, 11 and 2 isolated colonies, respectively. Zone 4 contains no colonies.

In row D, zones 1, 2 and 3 show a count of 115 isolated colonies, 14 isolated colonies and 1 isolated colony, respectively. Since the load is quite low, zones 4 and 5 do not contain any bacteria.

In this example, a close relationship is thus observed between the bacterial load of the suspension and the first countable zone. A shift in the first countable zone in fact appears as the microbial load increases by a factor of 10. The distribution is presented in table 1 below:

TABLE 1

| Theoretical | First | Result of |
|---|---|---|
| load | countable | counting this |
| (CFU/dish) | zone | zone |
| 80-130 | 1 | 115 |
| 800-1300 | 2 | 113 |
| 8000-13 000 | 3 | 116 |
| 80 000-130 000 | 4 | 67 |

It thus appears that a mathematical model can be readily produced for making it possible to obtain, on the basis of the reading of one or more countable zone(s), a relatively precise evaluation of the initial bacterial load of the solution.

### Example 3

### Evaluation of the Solution Volume Distribution Factor by Means of the Jump Spreading Method

### Procedure:

Starting from a solution calibrated at a theoretical bacterial load of 1000 CFU/ml, 100 μl of solution are deposited in the depositing zone and spread with the applicator used on the PREVI™ Isola system, while optionally performing jumps. The number of jumps performed is between 5 and 8. After incubation, a count is performed on each of the zones delimited by the jumps. The results are collated in FIG. 4. Row A corresponds to a spreading without jumps. Rows B, C, D and E correspond, respectively, to spreadings with 5, 6, 7 and 8 jumps.

### Results:

The number of isolated colonies for each zone is reported in table 2 below:

TABLE 2

| | Number of CPU/zone | | | | | | | | |
|---|---|---|---|---|---|---|---|---|---|
| | Zone | Zone | Zone | Zone | Zone | Zone | Zone | Zone | Total |
| | 8 | 7 | 6 | 5 | 4 | 3 | 2 | 1 | CFU |
| Row A | 79 | | | | | | | | 79 |
| Row B | — | — | — | 0 | 0 | 1 | 14 | 112 | 127 |
| Row C | — | — | 0 | 0 | 1 | 2 | 18 | 115 | 146 |
| Row D | — | 0 | 0 | 1 | 0 | 2 | 13 | 68 | 84 |
| Row E | 0 | 0 | 0 | 0 | 0 | 1 | 16 | 68 | 85 |

By taking the distribution of the bacteria in the suspension to be uniform, it is thus possible to determine the volume of suspension spread over each zone, by performing a simple rule of three. The results are collated in table 3 below:

TABLE 3

| | Volume of suspension (μl)/zone | | | | | | | | Total |
|---|---|---|---|---|---|---|---|---|---|
| | Zone | Zone | Zone | Zone | Zone | Zone | Zone | Zone | volume |
| | 8 | 7 | 6 | 5 | 4 | 3 | 2 | 1 | (μl) |
| Row A | 100 | | | | | | | | 100 |
| Row B | — | — | — | 0 | 0 | 0.787 | 11.02 | 88.19 | 100 |
| Row C | — | — | 0 | 0 | 0.685 | 1.37 | 12.33 | 78.77 | 100 |
| Row D | — | 0 | 0 | 1.19 | 0 | 2.381 | 15.48 | 80.95 | 100 |
| Row E | 0 | 0 | 0 | 0 | 0 | 1.176 | 18.82 | 80 | 100 |
| Average volume (μl) | | | | 1.19 | 0.685 | 1.429 | 14.41 | 81.98 | 100 |
| Rows A to E | | | | | | | | | |

The analysis of the results obtained below shows a certain uniformity in the distribution of the volumes. Specifically, taking into account the fact that the spreading is performed manually, and consequently with a limited reproducibility, a substantially similar suspension volume distribution profile is observed in zones 1 to 3. Thus, in zone 1, which is the zone where 100 μl of suspension are deposited, approximately 80% of the initial volume remains. In zone 2, the volume deposited is about 15% of the initial volume. Finally, in zone 3, it is between 1% and 2% of the volume which is deposited. The values thus obtained from one zone to the other are quite close to a logarithmic distribution. In other words, from one zone to the next, the number of colonies is approximately divided by ten.

By combining data for distribution of the solution volumes per zone, and the corresponding count values, it is possible, by means of a simple mathematical approach, to design a calculation model which makes it possible to evaluate the initial microbial load present in the base sample. Taking into account the improvement that can, moreover, be provided by the automation of the inoculation, this method would make it possible, by means of a single inoculation, to accurately count the microbial load of a solution over a wide microbial load range.

1. A method for isolating microorganisms on an agar culture medium, comprising the steps consisting in:
depositing, on said agar culture medium, a predetermined volume of a sample to be analyzed optionally containing said microorganisms or of a suspension of said microorganisms,
applying an inoculating means onto said agar culture medium in contact with the volume of sample or of suspension,
moving the inoculating means so as to totally or partially spread the volume of sample or of suspension over the surface of the agar culture medium, the movement of said inoculating means being discontinuous such that, during this movement, the contact between said inoculating means and the surface of the agar culture medium is interrupted and re-established at least once, creating successive spreading segments and resulting in a depletion of the inoculating means in terms of sample or of suspension, and
incubating said agar culture medium under conditions enabling the growth of microorganisms.

2. A method for counting microorganisms on an agar culture medium, comprising the steps consisting in:
depositing, on said agar culture medium, a predetermined volume of a sample to be analyzed optionally containing said microorganisms or of a suspension of said microorganisms,
applying an inoculating means onto said agar culture medium in contact with the volume of sample or of suspension,
moving the inoculating means so as to totally or partially spread the volume of sample or of suspension over the surface of the agar culture medium, the movement of said inoculating means being discontinuous such that, during this movement, the contact between said inoculating means and the surface of the agar culture medium is interrupted and re-established at least once, creating successive spreading segments and resulting in a depletion of the inoculating means in terms of sample or of suspension,
incubating said agar culture medium under conditions enabling the growth of microorganisms, and
counting the microorganism colonies present at the surface of the agar culture medium.

3. The method as claimed in claim 1, in which the inoculating means has a multitude of surfaces of contact with said culture medium.

4. The method as claimed in claim 1, in which the inoculating means has a single surface of contact with said culture medium.

5. The method as claimed in claim 1, in which the movement of the inoculating means is a rectilinear movement.

6. The method as claimed in claim 1, in which the movement of the inoculating means is a curvilinear movement.

7. The method as claimed in claim 1, implemented by means of an automated system.

8. The method as claimed in claim 1, in which the number of times the contact between said inoculating means and the surface of the agar culture medium is interrupted and re-established is between 2 and 6 times.

9. The method as claimed in claim 1, in which the volume deposited on the agar culture medium is between 10 and 1000 μl.

10. The method as claimed in claim 1, in which the spreading segments are variable in length.
